(19) United States
(12) Patent Application Publication
(10) Pub. No.: US 2026/0347887 A1
(43) Pub. Date: Nov. 5, 2026

(54) C2-C9 FATTY ACIDS IN ETHANOL PRODUCTION

(71) Applicant: Zinpro Corporation, Eden Prairie, MN (US)

(72) Inventors: PETER A. STARK, Inver Grove Heights, MN (US); MICHAEL THOMAS SOCHA, ROGERS, MN (US); CORY SHAWN KENDING, CHAMPLIN, MN (US)

(21) Appl. No.: 19/664,522

(22) Filed: Apr. 30, 2026

Related U.S. Application Data
(60) Provisional application No. 63/798,300, filed on May 1, 2025.
(60) Provisional application No. 64/001,434, filed on Mar. 10, 2026.

(51) Int. Cl. C12P 7/06

(52) U.S. Cl. CPC C12P 7/06

(57) ABSTRACT

This disclosure relates to the addition of C2 to C9 fatty acids, salts, or combinations thereof to the yeast fermentation step in ethanol production to increase the yield of ethanol produced. The fermentation step in ethanol production will include a yeast and a mash, the mash comprising a biofuel feedstock.

### CROSS REFERENCE TO RELATED APPLICATIONS

This application claims priority under 35 U.S.C. § 119(e) to provisional patent application U.S. Ser. No. 63/798,300, filed May 1, 2025 and U.S. Ser. No. 64/001,434, filed Mar. 10, 2026; each provisional patent application is hereby incorporated by reference in its entirety herein, including without limitation: the specification, claims, and abstract, as well as any figures, tables, appendices, or drawings thereof.

### TECHNICAL FIELD

The present disclosure relates generally to the addition of C2 to C9 fatty acids, salts thereof, and combinations thereof to the fermentation step in ethanol production to increase the yield of ethanol produced.

### BACKGROUND

The background description provided herein gives context for the present disclosure. Work of the presently named inventors, as well as aspects of the description that may not otherwise qualify as prior art at the time of filing, are neither expressly nor impliedly admitted as prior art.

Ethanol production from biofuels involves converting biomass, such as corn, sugarcane, or cellulosic materials, into fuel-grade alcohol through a multi-step process. First, the biomass is pre-treated to break down complex carbohydrates and starches into simpler sugars. This can be accomplished through milling, enzymatic hydrolysis, or chemical treatments. The simpler sugars are fermented by yeast which metabolize the sugar into ethanol and carbon dioxide. The fermentation step may also include enzymatic hydrolysis. After fermentation, solids are separated from the liquids. Ethanol is separated from the liquid via distillation, where it is heated to evaporate and condense into a concentrated form. Finally, the ethanol can be dehydrated to remove residual water, yielding fuel-grade ethanol suitable for blending with gasoline or use in flex-fuel vehicles. The process may vary depending on the feedstock with cellulosic ethanol requiring more advanced pretreatment due to the tougher plant fibers.

Ethanol produced from biofuels is a critical renewable energy source that plays a significant role in reducing greenhouse gas emissions and enhancing energy security. As a cleaner-burning alternative to fossil fuels, ethanol can be blended with gasoline to power vehicles, lowering carbon dioxide and particulate emissions, thus mitigating climate change impacts. Moreover, biofuels are derived from abundant and/or renewable feedstocks reducing reliance on finite petroleum reserves and supporting agricultural economies.

Furthermore, ethanol produced from biofuels has important applications beyond vehicle fuel, for example as an industrial or food-grade solvent, for beverage production, in pharmaceuticals, in cosmetics, and as a raw material in the production of plastics and rubber.

Accordingly, there is a continued need for methods that increase fermentation efficiency in ethanol production.

### BRIEF SUMMARY OF PREFERRED EMBODIMENTS

The following objects, features, advantages, aspects, and/or embodiments, are not exhaustive and do not limit the overall disclosure. No single embodiment need provide each and every object, feature, or advantage. Any of the objects, features, advantages, aspects, and/or embodiments disclosed herein can be integrated with one another, either in full or in part. Thus, this summary is not intended to identify key features or essential features of the claimed subject matter, nor is it intended to be used as an aid in determining the scope of the claimed subject matter.

In a preferred embodiment, a method of ethanol production comprising adding a C2 to C9 fatty acid to a fermentation process comprising a yeast and a mash; the mash comprising a biofuel feedstock, is described herein.

The foregoing general description of the illustrative embodiments and the following detailed description thereof are merely exemplary aspects of the teachings of this disclosure and are not restrictive.

### DETAILED DESCRIPTION

The present disclosure is not to be limited to that described herein. Mechanical, electrical, chemical, procedural, and/or other changes can be made without departing from the spirit and scope of the present disclosure. No features shown or described are essential to permit basic operation of the present disclosure unless otherwise indicated.

The present disclosure encompasses aspects and/or embodiments not expressly disclosed but which can be understood from a reading of the present disclosure, including at least: (a) combinations of disclosed aspects and/or embodiments and/or (b) reasonable modifications not shown or described.

Unless defined otherwise, all technical and scientific terms used above have the same meaning as commonly understood by one of ordinary skill in the art to which embodiments of the present disclosure pertain.

The terms “a,” “an,” and “the” include both singular and plural referents.

The term “or” is synonymous with “and/or” and means any one member or combination of members of a particular list.

Numeric ranges recited within the specification are inclusive of the numbers within the defined range. Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numerical values within that range, for example, 1, 2, 3, 4, 5, and 6, and decimals and fractions, for example, 1.2, 3.8, 1½, and 4¾ This applies regardless of the breadth of the range.

The term “about,” as used herein, refers to variations in size, distance or any other types of measurements that can be resulted from inherent heterogeneous nature of the measured objects and imprecise nature of the measurements itself, including, but not limited to, concentration, density, mass, molecular weight, odor, percentages, ratios, time, temperature, volume, pH, and weight. The term “about” also encompasses variation in the numerical quantity that can occur, for example, through typical measuring or handling procedures in the real world; through inadvertent error in these procedures; through differences in the manufacture, source, or purity of the ingredients used to make the device or carry out the methods, and the like. Whether or not modified by the term “about”, the claims include equivalents to the quantities.

The “invention” is not intended to refer to any single embodiment of the particular invention but encompass all possible embodiments as described in the specification and the claims. The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

The term “substantially” refers to a great or significant extent. “Substantially” can thus refer to a plurality, majority, and/or a supermajority of said quantifiable variables, given proper context.

The term “generally” encompasses both “about” and “substantially.”

The term “configured” describes structure capable of performing a task or adopting a particular configuration. The term “configured” can be used interchangeably with other similar phrases, such as constructed, arranged, adapted, manufactured, and the like.

Terms characterizing sequential order, a position, and/or an orientation are not limiting and are only referenced according to the views presented.

The present disclosure is related to increasing ethanol produced from biofuels by adding a C2-C9 fatty acid, salts thereof, or combination thereof during fermentation of the biofuel.

### Ethanol Fermentation

As used herein, ethanol fermentation refers to the process that utilizes yeast to convert sugars derived from a feedstock into ethanol and carbon dioxide.

As used herein “feedstock” refers to a biomass comprising sugar, starch, and/or cellulosic materials. Examples of a feedstock as used herein include, but are not limited to, corn, wheat, sugarcane, sugar beet, sorghum, straw, wood, switchgrass, miscanthus, corn stover, rice straw, bagasse, crop residues, wood residues, grass, algae and the like. Most preferred feedstock comprises corn, wheat, sugarcane, sugar beet, sorghum, or combinations thereof.

In an embodiment, ethanol fermentation comprises preparation or pretreatment of feedstock to modify the feedstock to make its sugars more accessible to enzyme hydrolysis. Any pretreatment as known in the art can is encompassed herein. In an embodiment, the pretreatment comprises mechanically reducing the size of the feedstock by mechanical grinding and/or milling. In an embodiment the pretreatment comprises denaturing and/or disrupting the cell wall for example by irradiation with microwaves. In an embodiment, the pretreatment comprises combining the feedstock with an acid or an alkaline solution to dissolve hemicellulose and/or loosen the cell wall structure. In an embodiment, the pretreatment comprises using oxygen or air and a catalyst (e.g. water or hydrogen peroxide) to oxidize lignin making cellulose more accessible. In an embodiment, the pretreatment comprises fungal pretreatment wherein fungi is used to degrade lignin and/or cellulose. In an embodiment, the pretreatment comprises utilizing steam to disrupt a cell wall structure.

In an embodiment, the ethanol fermentation method as described herein comprises mixing the pretreated feedstock with water forming a mash.

In an embodiment, the ethanol fermentation method as described herein comprises saccharification of the feedstock, pretreated feedstock, and/or mash. Any saccharification method as known in the art is encompassed herein. In an embodiment, ethanol fermentation comprises saccharification of the pretreated feedstock and/or mash. In an embodiment, saccharification comprises adding enzymes to the feedstock and/or pretreated feedstock and/or mash to break down cellulose and/or starch. Any enzyme as known in the art can be utilized herein, including for example an alpha amylase, an arabinofuranosidase, a cellulase, a beta-amylase, a glucoamylase, a gluco-alpha amylase, a protease, a xylanase, or combination thereof. In an embodiment, the enzyme comprises alpha amylase. In an embodiment, the enzyme comprises gluco-alpha amylase. In an embodiment, saccharification of the feedstock is at a temperature of from about 30 °C to about 90 °C, or from about 50 °C to about 90 °C or from about 80 °C to about 90 °C. In an embodiment, the saccharification is from about 60 minutes to about four hours, or from about 60 minutes to about two hours, or from about 80 minutes to about 100 minutes. In an embodiment, the pH of the feedstock, pretreated feedstock, and/or mash is adjusted to about 4.0 to about 6.0, from about 5.0 to about 6.0, or from about 5.0 to about 5.5 prior to saccharification. Any method of adjusting pH as known in the art is encompassed herein. In an embodiment, the method comprises adjusting the pH with an acid or a base to achieve a desired pH. In an embodiment, the method comprises adjusting the pH with 3M HCl. In an embodiment, the ethanol fermentation method as described herein comprises mixing the saccharified feedstock or pretreated feedstock with water forming a mash.

As known in the art, the use of a pretreatment and the type of pretreatment will be influenced by the type of biomass and its composition. As known in the art, the use of saccharification and the saccharification factors (temperature, time, pH, choice of enzyme, etc.) will be influenced by the type of biomass, the specific carbohydrate targeted, and any pretreatment of the biomass.

As used herein, a “mash” is the mixture that is fermented. A mash may comprise a feedstock, a pretreated feedstock, a saccharified feedstock, a saccharified pretreated feedstock, and combinations thereof.

In an embodiment, the ethanol fermentation method as described herein comprises adding a yeast to the mash and fermenting the sugars and starches in the mash. In an embodiment, the ethanol fermentation method as described herein comprises adding a C2, C3, C4, C5, C6, C7, C8, and/or C9 fatty acid, salt thereof, or combinations thereof to the mash during fermentation. In a preferred embodiment, the ethanol fermentation method as described herein comprises adding two or more C2, C3, C4, C5, C6, C7, C8, and/or C9 fatty acids, salts thereof, or combinations thereof. Applicants have found that adding C2 to C9 fatty acid, salt thereof, or combinations thereof beneficially increases ethanol production. The fatty acid can be added to the mash before or after the yeast. In a preferred embodiment, the fatty acid and yeast are added to the mash simultaneously.

In a preferred embodiment, from about 0.009% to about 5% of fatty acid, salt thereof, or combinations thereof is added to the mash on a weight by weight basis relative to the dry weight feedstock; more preferably 0.01 to about 1%, still more preferably 0.010% to about 0.5% of the fatty acid to the mash on a weight by weight basis relative to the dry weight feedstock. In a preferred embodiment, from about 0.009% to about 5% of two or more fatty acids, salts thereof, or combinations thereof. For example, the two or more fatty acids or salts thereof may individually be less than about 0.009%, but combined are at least about 0.009%, about 0.01% to about 1%, or about 0.01% to about 0.5% weight by weight basis relative to the dry weight feedstock.

As used herein, a “C6 yeast” is a yeast that ferments C6 sugars like glucose and fructose. In an embodiment, the method utilizes a C6 yeast. Any yeast strain that ferments C6 sugars like glucose and fructose as known in the art is encompassed herein. Examples of C6 yeasts include Saccharomyces cerevisiae (bakers' yeast). In an embodiment, the yeast utilized in the methods described herein comprises Saccharomyces cerevisiae, strains thereof, and/or derivatives thereof.

As used herein, a “C5 yeast” is a yeast that ferments C5 sugars like xylose and arabinose. In an embodiment, the method utilizes a C5 yeast. Any C5 yeast as known in the art is encompassed herein. Examples of C5 yeasts includes advanced strains of Saccharomyces cerevisiae. An example of a C5 yeast includes C5 FUEL™ from Lallemand.

In an embodiment, the yeast utilized in the methods described herein comprises a combination of C5 and C6 yeasts. An example of such a yeast is CELLERITY® 1.0 from Novonesis.

In an embodiment, the ethanol fermentation method as described herein comprises adding nitrogen and/or a nitrogen source to the mash. Any nitrogen or nitrogen sources as known in the art are encompassed herein. In an embodiment, the nitrogen source comprises ammonium ions, urea, peptone, amino acids, peptides, proteins, biological protein sources, and the like.

In an embodiment, the ethanol fermentation method as described herein comprises maintaining the temperature of the mash for example, preferably at about 10 °C to about 45 °C, more preferably at about 20 °C to about 40 °C, and most preferably about 30 °C to about 35 °C. In an embodiment, the fermentation takes from several hours to several days. In an embodiment, the fermenting step is about 24 hours to about 96 hours.

In an embodiment, an enzyme is added to the mash for simultaneous saccharification and fermentation. Any enzyme as known in the art can be utilized herein for example cellulases and amylases. In an embodiment, the enzyme comprises alpha amylase. In an embodiment, the enzyme comprises gluco-alpha amylase.

In an embodiment the ethanol fermentation method as described herein comprises separating the solids from the liquids (the fermentation broth) after fermentation is complete.

The composition of the solids depends on the feedstock. For example, sugarcane fermentation yields bagasse while corn fermentation yields distillers' grain. These solids can be used for various purposes for example animal feed. In conventional fermentation methods, without the addition of a C2-C9 fatty acid, fermented byproducts (like distillers' grain) naturally comprise isoacids, and when fed to ruminants can aid in digestion. Applicants have surprisingly found that adding additional specific C2-C9 fatty acids like isoacids to the mash during fermentation can improve ethanol production.

The fermentation broth comprises ethanol and other compounds. In embodiments, the fermentation broth is distilled to separate and purify the ethanol. The ethanol fermentation methods as disclosed herein increase the amount of ethanol in the fermentation broth as compared to an ethanol fermentation method that does not comprise adding a C2 to C9 fatty acid during fermentation. In embodiment, the methods as described herein increase ethanol production by about 2% to about 25% per mmol acid added depending on the specific acid.

### C2 to C9 Fatty Acids

As used herein, a “C2 to C9 fatty acid” refers to acids with 2 to 9 carbons and a carboxyl group that can readily dissolve, melt, and/or remain liquid at room temperature. C2 to C9 fatty acids include acetic acid, propionic acid, butyric acid, valeric acid, hexanoic acid, heptanoic acid, octanoic acid, nonanoic acid, isobutyric acid, 2-methylbutyric acid, isovaleric acid, isoheptanoic acid, isooctanoic acid, isononanoic acid, salts, and mixtures thereof. Also within the meaning of C2 to C9 fatty acid are the corresponding structural isomers including, but not limited, to 2-methylpentanoic acid, 3-methylpentanoic acid, 4-methylpentanoic acid, 2,2-dimethylbutanoic acid, 2,3-dimethylbutanoic acid, 3,3-dimethylbutanoic acid, 2-ethylbutanoic acid, 2-ethylpentanoic acid, 3-ethylpentanoic acid, 2-methylhexanoic acid, 3-methylhexanoic acid, 4-methylhexanoic acid, 5-methylhexanoic acid, 2-ethylhexanoic acid, 2-methylheptanoic acid, 3-methylheptanoic acid, 4-methylheptanoic acid, 5-methylheptanoic acid, 6-methylheptanoic acid, 2-ethyloctanoic acid, 3-ethyloctanoic acid, 2,2-dimethylheptanoic acid, 3,5-dimethylheptanoic acid, 4-methyloctanoic acid, and salts thereof. Also included within the meaning of C2 to C9 fatty acid is any salt thereof including acetate, propionate, butyrate, isobutyrate, 2-methyl butyrate, valerate, isovalerate, hexanoate, heptanoate, octanoate, and nonanoate, which may be solid at room temperature but can readily dissolve in a liquid at room temperature. Also included within the meaning of C2 to C9 fatty acid are salts thereof, including, but not limited to, ammonium salts, alkali metal salts, alkaline metal salts, transition metal salts, or mixtures thereof; preferred salts of the C2-C9 fatty acids comprise ammonium salts, potassium salts, calcium salts, sodium salts, magnesium salts, zinc salts, iron salts, and/or manganese salts. Preferred C2 to C9 fatty acids are acetic acid, propionic acid, butyric acid, valeric acid, hexanoic acid, heptanoic acid, octanoic acid, nonanoic acid, salts thereof, and mixtures thereof.

In an embodiment, the C2 to C9 fatty acids comprise an ester group. In an embodiment, the C2 to C9 fatty acids comprise a hydroxy group.

In an embodiment, the C2 to C9 fatty acids do not include esters thereof or hydroxys thereof.

### Preferred Embodiments

The methods disclosed herein are further demonstrated by the following numbered example embodiments; these embodiments are not restrictions on the methods that can be employed but are provided as non-limiting examples.
1. A method of ethanol production comprising: adding a C2 to C9 fatty acid to a fermentation process comprising a yeast and a mash; the mash comprising a biofuel feedstock.
2. The method of embodiment 1 wherein the C2 to C9 fatty acid comprises acetic acid, propionic acid, butyric acid, valeric acid, hexanoic acid, isobutyric acid, 2-methylbutyric acid, isovaleric acid, heptanoic acid, octanoic acid, nonanoic acid, salts thereof, isomers thereof, and mixtures of the foregoing.
3. The method of any one of embodiments 1-2 wherein the C2 to C6 fatty acid comprises an ammonium salt, a potassium salt, a magnesium salt, a calcium salt, a sodium salt, a zinc salt, an iron salt, a manganese salt, or combinations thereof of the fatty acid.
4. The method of any one of embodiments 1-3, wherein the C2 to C9 fatty acid comprises a combination of isobutyric acid, 2-methylbutyric acid, isovaleric acid, and/or valeric acid.
5. The method of any one of embodiments 1-3, wherein the C2 to C9 fatty acid comprises two or more of acetic acid, propionic acid, butyric acid, valeric acid, hexanoic acid, isobutyric acid, 2-methylbutyric acid, isovaleric acid, heptanoic acid, octanoic acid, nonanoic acid, or salts thereof.
6. The method of any one of embodiments 1-5, wherein the mash comprises corn, wheat, sugarcane, sugar beet, sorghum, straw, wood, switchgrass, miscanthus, corn stover, rice straw, bagasse, crop residues, wood residues, grass, algae, or combinations thereof.
7. The method of any one of embodiments 1-6, further comprising the step fermenting a sugar source within the mash to produce a fermentation broth comprising ethanol.
8. The method of any one of embodiments 1-7, wherein the yeast comprises Saccharomyces cerevisiae.
9. The method of any one of embodiments 1-8, wherein the yeast comprises a C6 yeast.
10. The method of any one of embodiments 1-8, wherein the yeast comprises a C5 yeast.
11. The method of any one of embodiments 1-9, wherein the yeast comprises a combination of a C5 and a C6 yeast.
12. The method of any one of embodiments 1-11, wherein the method comprises adding from about 0.009% to about 5% of the C2 to C9 fatty acid to the mash on a weight by weight basis relative to the dry weight feedstock.
13. The method of any one of embodiments 1-12, wherein the method comprises adding from about 0.010% to about 5% of the C2 to C9 fatty acid to the mash on a weight by weight basis relative to the dry weight feedstock.
14. The method of any one of embodiments 1-13, wherein the method comprises adding from about 0.01% to about 10% of the C2 to C9 fatty acid to the mash on a weight by weight basis relative to the dry weight feedstock.
15. The method of any one of embodiments 1-13, wherein the method comprises adding from about 0.01% to about 0.5% of the C2 to C9 fatty acid to the mash on a weight by weight basis relative to the dry weight feedstock.
16. The method of any one of embodiments 1-15, wherein the C2 to C9 fatty acid is a C3 to C6 fatty acid.
17. The method of any one of embodiments 1-16, wherein the method increases the amount of ethanol in the fermentation broth by from about 2% to about 25% as compared to a method that does not add a C2 to C9 fatty acid.
18. The method of any one of embodiments 1-17, further comprising adding an enzyme to the mash.
19. The method of embodiment 18, wherein the enzyme comprises an alpha amylase, an arabinofuranosidase, a cellulase, a beta-amylase, a glucoamylase, a gluco-alpha amylase, a protease, a xylanase, or combination thereof.
20. The method of any one of embodiments 1-19, further comprising adding a nitrogen source to the mash.
21. The method of embodiment 20, wherein the nitrogen source comprises ammonium ions, urea, peptone, amino acids, peptides, proteins, biological protein sources, or combinations thereof.
22. The method of any one of embodiments 1-21, wherein the mash is at a temperature of from about 10 °C to about 45 °C during fermentation.
23. The method of any one of embodiments 1-22, wherein the fermentation takes from about 12 hours to about 96 hours.
24. The method of any one of embodiments 1-23, wherein the biofuel feedstock is pretreated, saccharified, or a combination thereof.
25. The method of any one of embodiments 1-24, further comprising distilling the fermentation broth to obtain highly concentrated ethanol.

### Examples

Embodiments of the present disclosure are further defined in the following non-limiting Examples. It should be understood that these Examples, while indicating certain embodiments of the disclosure, are given by way of illustration only. From the above discussion and these Examples, one skilled in the art can ascertain the essential characteristics of this disclosure, and without departing from the spirit and scope thereof, can make various changes and modifications of the embodiments of the disclosure to adapt it to various usages and conditions. Thus, various modifications of the embodiments of the disclosure, in addition to those shown and described herein, will be apparent to those skilled in the art from the foregoing description. Such modifications are also intended to fall within the scope of the appended claims.

The following commercially available materials were utilized in the Examples: aqueous ammonium hydroxide solution (as the source of the ammonia group), urea, and various C2-C9 acids in the free acid and salt forms.

### Example 1—Ethanol Produced from Corn

In this Example, several different fatty acids were added during fermentation to evaluate the effect on ethanol production.

30 grams of corn ground to less than 800 μm was placed in a 250 mL flask and mixed with 125 mL deionized water. The pH of the mixture was adjusted to about 5.5 with 3M HCl. 20 μL of alpha amylase was added to the flask and mixed thoroughly. The flask was then weighed and incubated for 90 minutes at 85 °C. After incubation, the mixture was allowed to cool to room temperature. Fatty acid was added to the ambient mixture in amounts according to Table 1. At the same time, about 54 mg of urea equivalents, 20 μL of gluco-alpha-amylase and 0.5 mL of hydrated yeast were added while stirring at 200 rpm. The flask was weighed and then incubated for about 70 hours at 32 °C. During incubation, the weight of the flask was recorded twice per day until no more mass loss from CO₂ was reported. After the fermentation was complete, the solids and liquids were separated via filtration and analyzed for ethanol. For each fatty acid, multiple tests were run as indicated in Table 1. For each test, a control was conducted wherein no fatty acid was added to the mixture. The amount of ethanol with the fatty acid addition was compared to the control, and the average percent change in ethanol and average percent change per mmol fatty acid is recorded in Table 1. The percentage of fatty acid added is based on the following calculation:
30 g of Ground Corn at 15-18% moisture=~25.5-24.6 g of Dry Weight Corn
75 mg added to 25.5-24.6 g of dry weight corn=0.29-0.31% inclusion by percentage.

TABLE 1

| | | mg | mmols | % | Avg % | |
|---|---|---|---|---|---|---|
| | | Fatty | Fatty | Fatty | Change | Avg % |
| | # | Acid | Acid | Acid | in | Change/ |
| Fatty Acid | Runs | Added | Added | Added | Ethanol | mmol |
| Acetic acid | 8 | 75 | 1.25 | 0.3 | −3.40 | −2.73 |
| Propionic acid | 8 | 75 | 1.01 | 0.3 | 4.70 | 4.65 |
| Butyric acid | 12 | 75 | 0.85 | 0.3 | 0.63 | 2.69 |
| Isobutyric acid | 6 | 75 | 0.85 | 0.3 | 4.83 | 5.68 |
| 2-methyl butyric acid | 8 | 75 | 0.73 | 0.3 | 2.12 | 2.88 |
| Isovaleric acid | 2 | 75 | 0.73 | 0.3 | 8.60 | 11.71 |
| Valeric acid | 2 | 75 | 0.73 | 0.3 | 5.00 | 6.28 |

As can be seen from Table 1, acetic acid (a C2 carboxylic acid) is the only acid that deleteriously affected ethanol production. The remaining fatty acids all increased the amount of ethanol produced.

### Example 2—Combination of Fatty Acids

In this Example, combinations of fatty acids and their salts were evaluated according to the methodology outlined in Example 1. The results are shown in Table 2. When combinations are used, they are used in a molar ratio based upon the charge of the molecules, for example, ammonia and sodium are in a 1:1 ratio to the isoacids and magnesium and calcium are in a 1:2 ratio to the isoacids; excess metal (such as calcium) can be preferred to ensure all of the isoacid molecules are in the metal salt form. ISOFERM® is a proprietary blend of the calcium salt of branched-chain isoacids from ZINPRO® with a 2:1 ratio of isobutyric and 2 methyl butyric acid.

TABLE 2

| | | | | % | Avg % | |
|---|---|---|---|---|---|---|
| | | | | Fatty | Change | Avg % |
| | # | mg | mmols | Acid | in | Change/ |
| Combinations | Runs | Added | Added | Added | Ethanol | mmol |
| ISOFERM ® | 8 | 75 | 0.66 | 0.3 | 6.18 | 9.41 |
| ISOFERM ® | 2 | 75 | 0.61 | 0.3 | 11.45 | 18.89 |
| Ammonia Salt | | | | | | |
| calcium valerate - 2- | 4 | 75 | 0.62 | 0.3 | 10.00 | 16.15 |
| methyl butyric acid | | | | | | |
| Magnesium | 2 | 75 | 0.71 | 0.3 | 10.04 | 14.23 |
| isobutyrate - | | | | | | |
| isovaleric acid | | | | | | |
| potassium valerate - | 2 | 75 | 0.54 | 0.3 | 9.89 | 18.48 |
| isovaleric acid | | | | | | |
| 2-methylbutyric acid - | 2 | 75 | 0.73 | 0.3 | 10.60 | 14.43 |
| isovaleric acid | | | | | | |
| NH₃-isobutyrate | 2 | 75 | 0.71 | 0.3 | 9.31 | 13.05 |
| NH₃−2-methyl | 2 | 75 | 0.63 | 0.3 | 1.98 | 3.14 |
| butyrate | | | | | | |

As is shown in Table 2, the addition of combinations of C4 to C5 fatty acids, as well as ammonium, potassium, calcium, and magnesium fatty acid salts, during fermentation can significantly increase ethanol production.

### Example 3—Fatty Acid Esters and Hydroxys

In this Example, esters and hydroxys of fatty acids were evaluated according to the methodology outlined in Example 1. The results are shown in Table 3. For 3-hydroxy butyrate, the change in ethanol could not be recorded as the results of the two runs were inconsistent with one run increasing ethanol production and the other decreasing ethanol production.

TABLE 3

| | | | | % Fatty | Avg % | Avg % |
|---|---|---|---|---|---|---|
| Esters and | # | mg | mmols | Acid | Change in | Change/ |
| Hydroxys | Runs | Added | Added | Added | Ethanol | mmol |
| methyl isobutyrate | 2 | 75 | 0.73 | 0.3 | 5.7 | 7.76 |
| 3-hydroxybutyrate | 2 | 75 | 0.72 | 0.3 | | |
| isopropyl acetate | 2 | 75 | 0.73 | 0.3 | −13.92 | −18.95 |
| isobutyrate ethyl | 2 | 75 | 0.65 | 0.3 | −8.96 | −13.88 |
| ester | | | | | | |

As shown in Table 3, the addition of isoacids like methyl isobutyrate during fermentation increases production of ethanol, while the addition of hydroxys and esters of fatty acids did not.

### Example 4—Calcium Salts of Fatty Acids

In this Example, the calcium salts of various fatty acids and combinations of calcium salts of fatty acids were evaluated. The tests were conducted according to the methodology outlined in Example 1. The results are shown in Table 4. For combinations of fatty acids, the ratio of fatty acid salt to fatty acid salt is 1:1.

TABLE 4

| | | mg Fatty | mmols | % Fatty | % | |
|---|---|---|---|---|---|---|
| | # | Acid | Fatty Acid | Acid | Change in | % Change/ |
| Fatty Acid | Runs | Added | Added | Added | Ethanol | mmol |
| calcium propionate | 6 | 75 | 0.81 | 0.3 | 6.68 | 8.29 |
| calcium isobutyrate | 4 | 75 | 0.70 | 0.3 | 7.32 | 10.46 |
| calcium 2-methyl | 2 | 75 | 0.62 | 0.3 | 11.01 | 17.79 |
| butyric acid | | | | | | |
| calcium isobutyrate - | 8 | 75 | 0.66 | 0.3 | 6.18 | 9.41 |
| calcium 2-methyl | | | | | | |
| butyric acid | | | | | | |
| calcium 2-methyl | 4 | 75 | 0.62 | 0.3 | 10 | 16.15 |
| butyrate - calcium | | | | | | |
| valerate | | | | | | |
| calcium isovalerate | 4 | 75 | 0.66 | 0.3 | 10.04 | 15.21 |
| calcium hexanoate | 4 | 75 | 0.55 | 0.3 | 9.14 | 16.62 |

As is demonstrated in this Example, the addition of calcium salts of fatty acids and combinations of calcium salts during fermentation significantly increases ethanol production. Notably, the calcium salt of hexanoic acid beneficially increased ethanol production when the acid alone did not. Without being limited to a particular theory, calcium at relatively low levels is beneficial to yeast production by improving yeast growth and flocculation. Whereas, at high levels calcium may inhibit the invertase enzyme that breaks down disaccharides into glucose and fructose.

### Example 5—Ethanol Produced from Sugarcane Molasses

In this Example, the addition of fatty acids, combinations of fatty acids, or calcium salts of fatty acids, during the fermentation of sugarcane molasses was evaluated using the methodology outlined in Example 1 with one modification. Fermentation of sugarcane molasses does not require the addition of alpha amylase or the addition of gluco-alpha-amylase. Alpha amylase breaks down starch into simpler sugars which are then fermented by yeast. The addition of alpha amylase is therefore not required when fermenting sugarcane molasses which is primarily sucrose with smaller amounts of fructose and glucose. Examples 1-5 use a corn-based feedstock. The results are shown in Table 5.

TABLE 5

| | | mg Fatty | mmols | % Fatty | Avg % | Avg % |
|---|---|---|---|---|---|---|
| | # | Acid | Fatty Acid | Acid | Change in | Change/ |
| Fatty Acid | Runs | Added | Added | Added | Ethanol | mmol |
| ISOFERM ® | 16 | 75 | 0.66 | 0.3 | 5.08 | 7.69 |
| 2-methyl | 15 | 75 | 0.78 | 0.3 | 4.38 | 5.61 |
| butyric acid - | | | | | | |
| isobutyric | | | | | | |
| acid (1:2) | | | | | | |
| Isobutyric | 4 | 75 | 0.85 | 0.3 | 5.22 | 6.14 |
| acid | | | | | | |
| 2-methyl | 4 | 75 | 0.73 | 0.3 | 5.70 | 7.81 |
| butyric acid | | | | | | |
| Isovaleric | 4 | 75 | 0.73 | 0.3 | 8.48 | 11.62 |
| acid | | | | | | |
| hexanoic | 4 | 75 | 0.65 | 0.3 | 4.96 | 7.68 |
| acid | | | | | | |
| hexanoic | 4 | 75 | 0.22 | 0.3 | 7.97 | 37.07 |
| acid | | | | | | |
| heptanoic | 4 | 75 | 0.19 | 0.3 | 7.02 | 36.56 |
| acid | | | | | | |
| calcium | 4 | 75 | 0.62 | 0.3 | 10.03 | 16.17 |
| isovalerate | | | | | | |
| calcium 2- | 4 | 75 | 0.62 | 0.3 | 4.59 | 7.41 |
| methyl | | | | | | |
| butyrate | | | | | | |
| calcium | 4 | 75 | 0.70 | 0.3 | 2.81 | 4.02 |
| isobutyrate | | | | | | |

As shown in Table 5, the addition of C2 to C9 fatty acids, combinations of fatty acids, or salts thereof can significantly increase ethanol production from a feedstock of sugar molasses.

### Example 6—Ethanol Produced from Wheat

In this Example, the addition of fatty acid, combinations of fat acids, or fatty acid salts during the fermentation of wheat was evaluated using the methodology outlined in Example 1. The results are shown in Table 6.

TABLE 6

| | | mg Fatty | mmols | % Fatty | Avg % | Avg % |
|---|---|---|---|---|---|---|
| | # | Acid | Fatty Acid | Acid | Change in | Change/ |
| Fatty Acid | Runs | Added | Added | Added | Ethanol | mmol |
| ISOFERM ® | 3 | 75 | 0.66 | 0.3 | 2.06 | 3.14 |
| Isobutyric | 3 | 75 | 0.79 | 0.3 | 3.86 | 4.90 |
| acid - 2- | | | | | | |
| methyl | | | | | | |
| butyric acid | | | | | | |
| Isobutyric | 3 | 75 | 0.85 | 0.3 | 6.17 | 7.25 |
| acid | | | | | | |
| calcium 2- | 3 | 75 | 0.62 | 0.3 | 4.53 | 7.31 |
| methyl | | | | | | |
| butyrate | | | | | | |
| calcium | 3 | 75 | 0.70 | 0.3 | 5.01 | 7.15 |
| isobutyrate | | | | | | |
| Valeric acid - | 2 | 75 | 0.73 | 0.3 | 7.25 | 9.87 |
| 2-methyl | | | | | | |
| butyric acid | | | | | | |
| Isovaleric | 3 | 75 | 0.73 | 0.3 | 8.32 | 11.33 |
| acid | | | | | | |

As shown in Table 6, the addition of a C2-C9 fatty acid, combination of C2-C9 fatty acids, or C2-C9 fatty acid salts during the fermentation of wheat significantly increases the production of ethanol.

### Example 7—Ethanol Produced from Sorghum

In this Example, the addition of a fatty acid, combinations of fatty acids, or fatty acid salts during the fermentation of sorghum was evaluated using the methodology outlined in Example 1. The results are shown in Table 7.

TABLE 7

| | | mg Fatty | mmols | % Fatty | Avg % | Avg % |
|---|---|---|---|---|---|---|
| | # | Acid | Fatty Acid | Acid | Change in | Change/ |
| Fatty Acid | Runs | Added | Added | Added | Ethanol | mmol |
| ISOFERM ® | 3 | 75 | 0.66 | 0.3 | 2.23 | 3.39 |
| Isobutyric | 3 | 75 | 0.79 | 0.3 | 1.81 | 2.29 |
| acid - 2- | | | | | | |
| methyl butyric | | | | | | |
| acid | | | | | | |
| Isobutyric | 3 | 75 | 0.85 | 0.3 | 3.95 | 4.64 |
| acid | | | | | | |
| calcium 2- | 3 | 75 | 0.62 | 0.3 | 4.04 | 6.52 |
| methyl butyric | | | | | | |
| acid | | | | | | |
| calcium | 3 | 75 | 0.70 | 0.3 | 1.65 | 2.35 |
| isobutyrate | | | | | | |
| Valeric acid - | 3 | 75 | 0.73 | 0.3 | 5.43 | 6.48 |
| 2-methyl | | | | | | |
| butyric acid | | | | | | |
| Isovaleric acid | 3 | 75 | 0.73 | 0.3 | 6.12 | 8.34 |

As shown in Table 7, the addition of a C2-C9 fatty acid, combination of C2-C9 fatty acids, or C2-C9 fatty acid salts during the fermentation of sorghum significantly increases the production of ethanol.

### Example 8—Combination of Fatty Acids

In this Example, the addition of a fatty acid, or combinations of fatty acids were evaluated at 0.005%, 0.01%, and 0.1% weight by weight basis relative to the dry weight feedstock. The tests were conducted according to the methodology outlined in Example 1. The results are shown in Table 8. For combinations of fatty acids, the ratio is 1:1, 1:1:1, and 1:1:1:1.

TABLE 8

| | 0.005% | 0.01% | 0.1% |
|---|---|---|---|
| | Fatty | Fatty | Fatty |
| | Acid | Acid | Acid |
| Fatty Acid | Added | Added | Added |
| Isobutyric acid - 2-methyl butyric acid | — | −0.11 | 7.11 |
| Isovaleric acid - 2-methyl butyric acid | — | −0.55 | 9.40 |
| Isobutyric acid - isovaleric acid | −2.62 | 2.36 | 7.14 |
| 2-methyl butyric acid - valeric acid | −3.60 | 3.22 | 5.99 |
| Isobutyric acid - Valeric acid | −2.58 | 2.21 | 7.55 |
| Isovaleric acid - Valeric acid | −7.51 | 2.67 | 6.71 |
| Isobutyric acid - 2-methyl butyric acid - | 1.17 | 1.16 | 9.22 |
| Isovaleric acid | | | |
| Isobutyric acid - 2-methyl butyric acid - | — | 0.68 | 5.29 |
| Valeric acid | | | |

As can be seen from Table 8, the addition of combinations of C2-C9 fatty acids at least 0.01% increased the production of ethanol, with 0.1% significantly increasing the production of ethanol.

### Example 9—Fatty Acid Amounts

In this Example, the addition of a C2-C9 fatty acid, or combinations of C2-C9 fatty acids were evaluated at from 0.005% to 1.5% weight by weight basis relative to the dry weight feedstock. The tests were conducted according to the methodology outlined in Example 1 except that variable ground corn was replaced with ground corn flour to reduce variability that may be imparted by the corn source. The results are shown in Table 9. For the combination of isobutyrate and 2-methyl butyrate, the ratio is 1:1.

TABLE 9

| Amount | | | | | | | | | |
|---|---|---|---|---|---|---|---|---|---|
| of Fatty | | | | | 2- | | | Isobutyric - | |
| Acid | | | | | Methyl | | | 2-methyl | |
| Added | Acetic | Propionic | Butyric | Isobutyric | butyric | Isovaleric | Valeric | butyric | Hexanoic |
| (%) | acid | acid | acid | acid | acid | acid | acid | acid | Acid |
| 0.005 | −0.5 | −0.71 | −2.18 | 0.06 | −1.02 | 0.02 | — | — | −2.4 |
| 0.01 | 2.2 | −3.62 | 0.31 | 4.06 | 1.93 | 2.14 | −0.37 | −0.11 | 6.1 |
| 0.05 | 1.2 | −0.34 | 10.69 | 5.56 | 7.36 | 6.41 | 7.65 | 5.34 | 17.4 |
| 0.1 | 1.6 | 0.98 | 11.85 | 7.63 | 7.69 | 7.52 | 7.17 | 7.11 | 17.3 |
| 0.15 | — | — | — | — | — | — | — | — | 20.4 |
| 0.2 | — | — | — | — | — | — | — | — | 19 |
| 0.25 | — | — | — | — | — | — | — | — | −11.4 |
| 0.3 | — | 8.9 | — | — | — | — | — | — | −44.6 |
| 0.5 | 1.5 | 8.02 | 13.05 | 10.29 | 9.46 | 10.81 | 6.58 | 7.67 | −87.7 |
| 0.675 | — | −2 | — | — | — | — | — | — | 0 |
| 0.75 | — | −14.1 | 4.5 | — | — | 13.6 | — | — | 0 |
| 0.875 | — | 0 | — | — | — | 0.04 | — | — | 0 |
| 1 | −48.5 | −45.5 | −27.5 | 5.57 | 6.2 | −5.14 | −87.63 | 9.75 | 0 |
| 1.25 | 0 | 0 | 0 | — | — | 0 | 0 | −20 | 0 |
| 1.5 | — | — | −91.7 | −60.5 | −91.7 | — | — | −75.2 | — |

As shown in Table 9, the addition of C2-C9 fatty acids, individually or in combination, increased the production of ethanol.

### Example 10—Medium Chain Fatty Acids

In this Example, the addition of a C6-C10 fatty acid, or combinations of C6-C10 fatty acids were evaluated at 0.005%, 0.01%, 0.05%, 0.1%, 0.15%, 0.2%, 0.25%, and 0.3% weight by weight basis relative to the dry weight feedstock. The tests were conducted according to the methodology outlined in Example 1. The results are shown in Table 10.

TABLE 10

| | 0.005% | 0.01% | 0.05% | 0.1% | 0.15% | 0.2% | 0.25% | 0.3% |
|---|---|---|---|---|---|---|---|---|
| | Fatty | Fatty | Fatty | Fatty | Fatty | Fatty | Fatty | Fatty |
| | Acid | Acid | Acid | Acid | Acid | Acid | Acid | Acid |
| Fatty Acid | Added | Added | Added | Added | Added | Added | Added | Added |
| Hexanoic Acid | −2.4 | 6.1 | 17.4 | 17.3 | 20.4 | 19 | −11.4 | −44.6 |
| Heptanoic Acid | — | 2.22 | 13.9 | 16.9 | — | — | — | −17.7 |
| Octanoic Acid | — | 4.07 | 16.55 | 17.59 | — | — | — | −2.2 |
| Nonanoic Acid | — | −0.18 | 13.56 | 14.13 | — | — | — | −2.8 |
| Decanoic Acid | — | — | — | — | — | — | — | −12.18 |

As can be seen from Table 10, the addition of C6-C9 fatty acids from 0.01%-0.2% increased the production of ethanol compared to those included at higher amounts (0.3%) and lower amounts (0.005%).

### Example 11—Combination of Fatty Acids

In this Example, the addition of a C2-C9 fatty acids were evaluated individually at 0.005% and 0.01%, weight by weight basis relative to the dry weight feedstock. Further, combinations of C2-C9 fatty acids were evaluated combined at 0.009%, with each C2-C9 fatty acid at 0.0045%, or 0.003% when three C2-C9 fatty acids were combined. The tests were conducted according to the methodology outlined in Example 1. The results are shown in Table 11.

TABLE 11

| | 0.005% Fatty | 0.009% Combined | 0.01% Fatty |
|---|---|---|---|
| Fatty Acid | Acid Added | Fatty Acid Added | Acid Added |
| Isobutyric Acid | 0.06 | 2.58 | 4.06 |
| Hexanoic Acid | −2.40 | | 6.10 |
| Butyric acid | −2.18 | 5.04 | 0.31 |
| Hexanoic Acid | −2.40 | | 6.10 |
| Isobutyric Acid | 0.06 | 4.07 | 4.06 |
| Butyric Acid | −2.18 | | 0.31 |
| Hexanoic Acid | −2.40 | | 6.10 |

As can be seen from Table 11, the combination of C2-C9 fatty acids at a combined amount of 0.009% increased the production of ethanol at higher amounts compared to the 0.005% individual C2-C9 fatty acids. This demonstrates a synergistic effect where the combination provided greater results than the sum of the fatty acids each on their own.

The above disclosure is intended to be illustrative and not exhaustive. This description will suggest many variations and alternatives to one of ordinary skill in this art. All these alternatives and variations are intended to be included within the scope of the following claims where the term “comprising” means “including, but not limited to”. Those familiar with the art may recognize other equivalents to the specific embodiments described herein which equivalents are also intended to be encompassed by the following claims.

What is claimed is:

1. A method of ethanol production comprising:
adding a C2 to C9 fatty acid to a fermentation process comprising a yeast and a mash; the mash comprising a biofuel feedstock.

2. The method of claim 1, wherein the C2 to C9 fatty acid comprises acetic acid, propionic acid, butyric acid, valeric acid, hexanoic acid, isobutyric acid, 2-methylbutyric acid, isovaleric acid, heptanoic acid, octanoic acid, nonanoic acid, salts thereof, isomers thereof, and mixtures of the foregoing.

3. The method of claim 1, wherein the C2 to C6 fatty acid comprises an ammonium salt, a potassium salt, a magnesium salt, a calcium salt, a sodium salt, a zinc salt, an iron salt, a manganese salt, or combinations thereof of the fatty acid.

4. The method of claim 3, wherein the C2 to C9 fatty acid comprises a combination of isobutyric acid, 2-methylbutyric acid, isovaleric acid, and/or valeric acid.

5. The method of claim 1, wherein the C2 to C9 fatty acid comprises two or more of acetic acid, propionic acid, butyric acid, valeric acid, hexanoic acid, isobutyric acid, 2-methylbutyric acid, isovaleric acid, heptanoic acid, octanoic acid, nonanoic acid, or salts thereof.

6. The method of claim 1, wherein the mash comprises corn, wheat, sugarcane, sugar beet, sorghum, straw, wood, switchgrass, miscanthus, corn stover, rice straw, bagasse, crop residues, wood residues, grass, algae, or combinations thereof.

7. The method of claim 1, further comprising the step fermenting a sugar source within the mash to produce a fermentation broth comprising ethanol.

8. The method of claim 1, wherein the yeast comprises Saccharomyces cerevisiae.

9. The method of claim 1, wherein the yeast comprises a C6 yeast, a C5 yeast, or a mixture thereof.

10. The method of claim 1, wherein the method comprises adding from about 0.009% to about 5% of the C2 to C9 fatty acid to the mash on a weight by weight basis relative to the dry weight feedstock.

11. The method of claim 10, wherein the method comprises adding from about 0.01% to about 1% of the C2 to C9 fatty acid to the mash on a weight by weight basis relative to the dry weight feedstock.

12. The method of claim 1, wherein the C2 to C9 fatty acid is a C3 to C6 fatty acid.

13. The method of claim 10, wherein the method increases the amount of ethanol in the fermentation broth by from about 2% to about 25% as compared to a method that does not add a C2 to C9 fatty acid.

14. The method of claim 1, further comprising adding an enzyme to the mash.

15. The method of claim 14, wherein the enzyme comprises an alpha amylase, an arabinofuranosidase, a cellulase, a beta-amylase, a glucoamylase, a gluco-alpha amylase, a protease, a xylanase, or combination thereof.

16. The method of claim 1, further comprising adding a nitrogen source to the mash.

17. The method of claim 16, wherein the nitrogen source comprises ammonium ions, urea, peptone, amino acids, peptides, proteins, biological protein sources, or combinations thereof.

18. The method of claim 1, wherein the mash is at a temperature of from about 10 °C to about 45 °C during fermentation; and wherein the fermentation takes from about 12 hours to about 96 hours.

19. The method of claim 1, wherein the biofuel feedstock is pretreated, saccharified, or a combination thereof.

20. The method claim 1, further comprising distilling the fermentation broth to obtain highly concentrated ethanol.
